(19) United States
(12) Patent Application Publication
(10) Pub. No.: US 2014/0366441 A1
(43) Pub. Date: Dec. 18, 2014

(54) VEGETATIVE PROPAGATION OF SOYBEAN PLANTS IN A HYDROPONIC ENVIRONMENT

(71) Applicant: Bayer Cropscience LP, Research Triangle Park, NC (US)

(72) Inventors: Nanfei Xu, Cary, NC (US); Alain Sailland, Raleigh, NC (US); Amy McCaskill, Apex, NC (US)

(21) Appl. No.: 14/376,933

(22) PCT Filed: Feb. 26, 2013
(86) PCT No.: PCT/US2013/027769

§ 371 Date: Aug. 6, 2014

Related U.S. Application Data
(60) Provisional application No. 61/603,574, filed on Feb. 27, 2012.

(51) Int. Cl. A01G 1/00

(52) U.S. Cl. CPC A01G 1/001
USPC 47/58.1 R

(57) ABSTRACT

The present invention is directed to a method of clonally propagating crop plant material in a hydroponic environment. The methods are useful for evaluating transgenic crop plants following transformation or expanding the number of genetically identical plants and seed generated therefrom. The methods comprise removing a part from a crop plant, such as a shoot or stem, and placing it in a hydroponic medium sufficient to support the development of one or more roots in the hydroponic medium. The new plant can then be grown under suitable conditions into a mature crop plant.

### CROSS REFERENCE TO RELATED APPLICATIONS

This application claims the benefit of U.S. Provisional Application No. 61/603,574, filed Feb. 27, 2012, the content of which is incorporated by reference in its entirety.

### FIELD OF THE INVENTION

This invention is related to the field of plant propagation, particularly vegetative propagation.

### BACKGROUND OF THE INVENTION

The process of developing a transgenic crop is considerably more complicated than the textbook description of identifying a gene of interest, inserting it into a crop plant, and multiplying the seed to obtain a new cultivar. This process for developing a commercial transgenic product can take 12 or more years and includes the resource-intensive steps of identification of a candidate gene, transformation and event production, greenhouse testing, molecular characterization, field testing and elite event selection, regulatory clearance, introgression into different genetic backgrounds, breeding and variety registration, and seed production (Devine (2005) Pest Management Science, 61(3):312-317). It is clear that the research community is generating many different transgenic plants, but many of these are not being developed and commercialized due to these constraints. Thus, improvements in the efficiency of this process could expand the number of GMO products on the market or accelerate the timeline from discovery to product launch.

### SUMMARY OF INVENTION

Provided herein is a method of clonally propagating agricultural crop plant material in a hydroponic environment. The methods are useful for evaluating transgenic crop plants following transformation or expanding the number of genetically identical plants and seed generated therefrom. The methods comprise removing a part (or “cutting”) from a crop plant, such as a primary or secondary shoot or stem, and placing it in a hydroponic medium sufficient to support the development of one or more roots in the hydroponic medium. The new plant can then be grown under suitable conditions into a mature plant.

The hydroponic medium may comprise various nutrients and/or plant growth hormones. In various embodiments, the hydroponic medium is contained within a translucent box containing a lid with an opening sufficient to support the part of the plant in an upright position in the hydroponic medium.

### BRIEF DESCRIPTION OF THE FIGURES

FIG. 1A shows the effects of fertilizer on root length from shoot cuttings of soybean plants; FIG. 1B shows the effects of NAA on root length from shoot cuttings of soybean plants.

FIG. 2 depicts examples of roots grown from soybean shoot cuttings.

FIG. 3 shows the effects of NAA on root number and root length of soybean shoot cuttings.

FIG. 4 shows the effects of shoot length on root number and root length of soybean shoot cuttings.

FIG. 5 depicts corn shoots 3 days (FIG. 5A) and 5 days (FIG. 5B) into the cloning process. Long roots appeared on every shoot.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. As used herein, the term “about”, when referring to a value or to an amount of mass, weight, time, volume, concentration or percentage is meant to encompass variations of ±20%, in another example ±10%, in another example ±5%, in another example ±1%, and in still another example ±0.1% from the specified amount, as such variations are appropriate to practice the presently disclosed subject matter. Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter. Unless defined otherwise, all technical and scientific terms used herein have the same meaning as commonly understood to one of ordinary skill in the art to which this disclosure belongs.

Many plant species, such as several fruits and ornamental plant species, are commonly reproduced by vegetative propagation (or “clonal propagation” or “vegetative reproduction”). Vegetative propagation is the ability of plants to reproduce without sexual reproduction, by producing new, genetically identical, plants from existing vegetative structures. The most common method of artificial vegetative propagation involves removal of parts (commonly referred to as “cuttings”) from the parent plant and placed in a suitable environment where they can grow into a whole new plant. Cutting takes advantage of the ability of plants to form adventitious roots under certain conditions, and the resulting plant is a clone of the parent plant.

The present invention describes methods for clonally propagating agricultural crop plants, particularly maize, sorghum, wheat, cotton, rice, soybean, sugarbeet, sugarcane, tobacco, barley, and oilseed rape crop plants. In various embodiments, the plants are clonally propagated in a hydroponic environment. In a hydroponic system, generally, plants are grown using mineral nutrients in an aqueous environment and without the aid of a purely soil base. In such a system, the root system of the plants must remain in a sufficiently aqueous environment in order to survive.

In various embodiments, the crop plants are genetically modified (or “transgenic”) plants. Transgenic plants are plants of which a heterologous gene has been stably integrated into its genome. The expression “heterologous gene” essentially means a gene which is provided or assembled outside the plant and when introduced in the nuclear, chloroplast or mitochondrial genome gives the transformed plant new or improved agronomic or other properties by expressing a protein or polypeptide of interest or by downregulating or silencing other gene(s) which are present in the plant.

One particular advantage of the present invention is that cuttings can be taken from T0 plants and cloned vegetatively. This process can significantly expand the amount of genetically identical plant material that can be evaluated from a single transformation event.

This process provides a significant improvement in the efficiency of the selection of a desirable transgenic line by providing a means to evaluate the single event, for example, under a variety of experimental conditions.

Similarly, homozygous T1 (or later) generation crop plants can be cloned to produce sufficient seed for planting and testing of subsequent generations. This method effectively accelerates the normal pipeline for transgenic plant selection and evaluation by a generation, thereby saving time and resources in the process.

The methods of the invention comprise removing a part (or “cutting”) from the cropplant for propagation. By plant “parts” is intended all above ground vegetative parts of crop plants such as primary or secondary shoots, leaf, stems, branches, and the like. The methods are useful for any portion of the plant from which adventitious roots can form in aqueous conditions.

In some embodiments, the plant part or cutting is a shoot. The shoot can be at least about 5 cm, at least about 10 cm, at least about 15 cm, at least about 20 cm, or larger. Plants can be cloned from V3 (3 trifoliate leaves) to R6 (seed filling) stages. In various embodiments, plants are cloned anywhere from stages V6 (6 trifoliate leaves) to R3 (beginning of pod formation). In some embodiments, later stage cloning is performed under long periods of light.

The cuttings obtained from the parent plant are placed in a hydroponic medium for root development. The term “hydroponic” refers to a method for cultivating plant material in a mineral nutrient solution rather than in soil. Many different types of hydroponic systems are available and the methods of the present invention are not limited to any particular type of system.

In some embodiments, the hydroponic system of the present invention comprises a vessel suitable for holding a sufficient volume of hydroponic medium to support the growth of adventitious roots from the crop plant cutting. For example, translucent or transparent containers capable of supporting the growth of the cutting in an upright position while partially submerged in the hydroponic medium can be used. In one embodiment, the container is a tissue culture box with a lid affixed thereto. The lid may contain an opening sufficient to physically support the cutting in the hydroponic medium. In various embodiments, the base of the cutting is submerged in the hydroponic medium while the top of the cutting extends above the opening of the lid. The cutting can be further supported by any suitable device such as a tube or other support structure placed in or near the lid opening.

The hydroponic medium suitable for the vegetative propagation of crop plants is an aqueous medium comprising one or more water-soluble nutrients sufficient to support growth and development, e.g. adventitious root development, of the cutting. In various embodiments, the nutrients are supplied as a water-soluble fertilizer. The fertilizer can be an all-purpose fertilizer suitable for use with the specific crop plant of interest. The fertilizer may further comprise, or the hydroponic medium may be further supplemented with, one or more additional nutrients such as, but not limited to, calcium nitrate, potassium nitrate, monobasic potassium phosphate, magnesium sulfate, potassium silicate, potassium sulfate, iron(III)chloride, ethylenediamine-N,N′-bis(2-hydroxyphenylacetic acid), ethylenediaminetetraacetic acid, diethylene triamine pentaacetic acid, manganese chloride, zinc chloride, boric acid, copper(II)chloride, sodium molybdate, magnesium nitrate, heptahydrate epsomite, and the like.

In some embodiments, the hydroponic medium comprises at least one plant growth hormone. In various embodiments, the plant growth hormone is an auxin, for example, 1-napthaleneacetic acid (NAA), indole-3-acetic acid (IAA), indole-3-butyric acid (IBA), indole-3-propionic acid (IPA) and 2,4-dichlorophenoxyacetic acid (2,4-D). In specific embodiments, the auxin is NAA. NAA can be added to the hydroponic medium at a final concentration of about 10-5 M, about 10-6 M, about 10-7 M, about 10-8 M, or about 10-9 M.

The plant material may be grown hydroponically in a controlled growth environment using conventional sunlight or using an artificial light source. The use of an artificial light source as a part of hydroponics allows for greater illumination over more hours than is possible by conventional sunlight. Accordingly plant growth, and therefore yields, can be significantly increased for a given amount of time. Thus, in various embodiments, the plants are exposed to at least about 16, at least about 17, 18, 19, 20, 21, or 22 hours of light per day at a temperature of about 26° C., about 27° C., about 28° C., about 29° C., or about 30° C., with corresponding dark periods at about 20° C., about 21° C., about 22° C., about 23° C., or about 24° C. In specific embodiments, the plants are exposed to light for about 20 hours per day a day at a temperature of about 28° C., with 4 hours per day at a temperature of about 22° C. Following sufficient root development from the shoots, the plants can be transferred to soil under standard conditions for growth and maturity.

Crop plants and cultivars which can be cloned according to the present invention include all plants which have genetic material which impart particularly advantageous, useful traits to these plants. For example, crop plants that can be cloned using the methods of the present invention may be resistant to one or more biotic stresses, i.e. said plants show a better defense against animal and microbial pests, such as against nematodes, insects, mites, phytopathogenic fungi, bacteria, viruses and/or viroids.

Crop plants and cultivars which can be cloned according to the present invention also include plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Crop plants and cultivars which can be cloned according to the present invention also include plants which are characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture Further yield traits include seed composition, such as carbohydrate content, protein content, oil content and composition, nutritional value, reduction in anti-nutritional compounds, improved processability and better storage stability.

Crop plants and cultivars which can be cloned according to the present invention also include plants which are herbicide-tolerant plants, i.e. plants made tolerant to one or more given herbicides such as glyphosate, glutamine synthetase inhibitor herbicides, hydroxyphenylpyruvatedioxygenase (HPPD) inhibitor herbicides, acetolactate synthase (ALS) inhibitor herbicides, and the like.

Crop plants and cultivars which can be cloned according to the present invention also include plants which are insect-resistant transgenic plants, i.e. plants made resistant to attack by certain target insects or other plant pests.

As would be understood by one of ordinary skill in the art upon a review of the present disclosure, numerous herbicide resistance, antibiotic resistance, disease resistance, pest/insect resistance, drought resistance, and selectable or scorable marker genes are known in the art, as are various methods of introducing said genes into a plant cell, regenerating tissues from said plant cell, and selecting for transformants.

The following examples are offered by way of illustration and not by way of limitation.

### EXPERIMENTAL EXAMPLES

### Example 1

Soybean shoots 10 to 15 cm long were cut from 2 similar plants and placed in 50 ml plastic tubes. One shoot was placed in each tube. The tubes contain 50 ml solutions according to the following design:

The base solution was either water or a 1:200 dilution of fertilizer stock solution A, B and C. Stock A contains 958.6 grams of 21-5-20 All Purpose from Jack's Professional, 133.8 grams of 15-0-15 Dark Weather (Jack's Professional) and 60.0 grams of 10-0-0 Magnitrate Special (Peter's Excel) in 2 liter of water. Stock B contains 239.6 grams of 15-5-25 Poinsettia Peat-Lite Special (Jack's Professional) in 2 liters of water. Stock C contains 60.0 grams of Epsom salt (magnesium sulfate) in 1.0 liter of water. NAA (1-Naphthaleneacetic acid, Sigma-Aldrich) was added to either water or fertilizer solutions to concentrations of 0, 10-9 M, 10-8 M and 10-7 M. There were 2 fertilizer (with or without) treatments×4 NAA treatments, totaling 8 treatments. The experiment was repeated 3 times, making a total of 24 tubes.

The shoots in the solutions were placed in a greenhouse with 20 hour light cycles at 28° C. and 4 hour night cycles at 22° C.

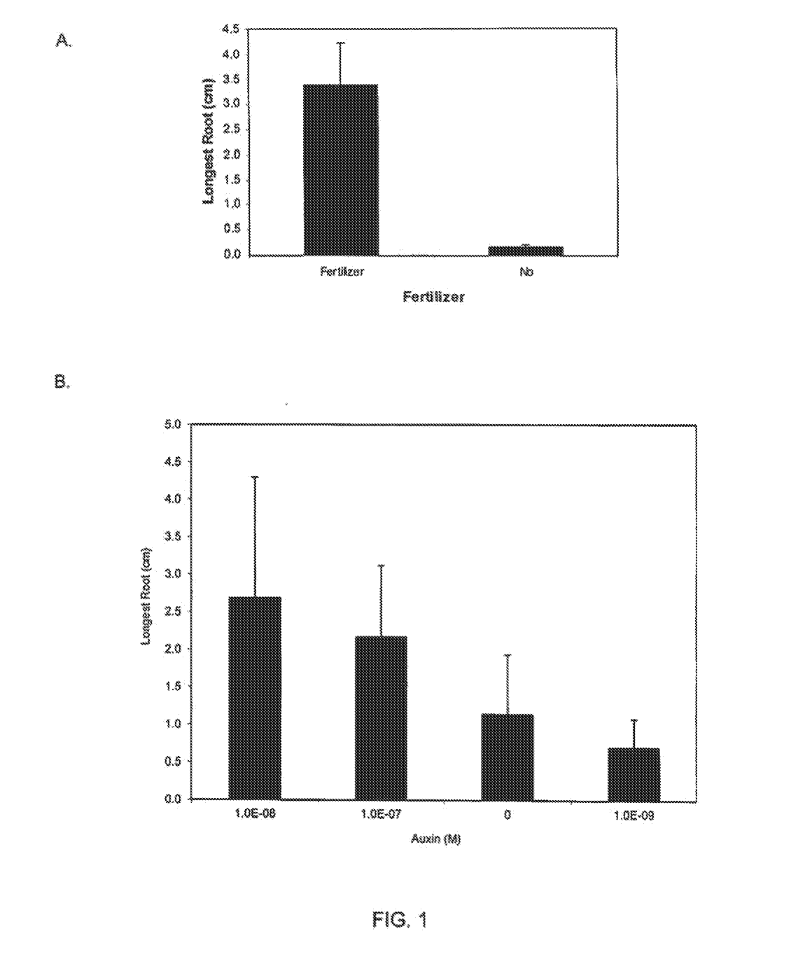

The shoots consumed water rapidly. The tubes had to be filled twice daily to avoid drying of the shoots. Some shoots were dry at some points, resulting in wilting and delay in root development. However, by day 12, all but one shoot developed roots. Most of the shoots had short roots but some developed long healthy roots. Fertilizer significantly promoted root growth (FIG. 1A) and NAA at 10-8 M and 10-7 M also showed positive effects on root growth (FIG. 1B).

### Example 2

Three NAA concentrations at 10-8 M, 10-7 M and 10-6 M with 5 cm, 10 cm and 15 cm long shoots were tested in replicate. Soybean shoot cuttings were taken from plants recovered from tissue culture. Tissue culture boxes (Vitro Desksel) were used to hold the solutions, and the lids with a 2.5 cm hole in the middle were used to hold the shoots with the help of a cylindrical sponge. NAA solution was obtained from PhytoTechnology Laboratories. Fertilizer was applied from the stock A, B, and C solutions described in Example 1.

Three separate 5-liter fertilizer solutions were made by mixing 10 ml each of fertilizer stock solution A, B and C in 4970 ml of tap water. NAA stock solution was added to achieve a final concentration of 10-8 M, 10-7 M and 10-6 M solution, respectively. Soybean shoot cuttings were inserted into the sponge roll, and placed through the lid of the tissue culture box. The shoots in the solutions were placed in a greenhouse with 20 hour light cycles at 27° C. and 4 hour night cycles at 21° C. Data was collected nine days after the shoots were placed in the boxes.

All the shoot cuttings wilted only slightly for the first few hours and the leaves then stayed fully expanded for the duration of the experiment. The shoots stayed green and continued to grow. Because of the larger reservoir of the box and the lid to prevent evaporation, the solution volume in the boxes remained high for several days; only some boxes that had larger shoots (15 cm) needed one refill.

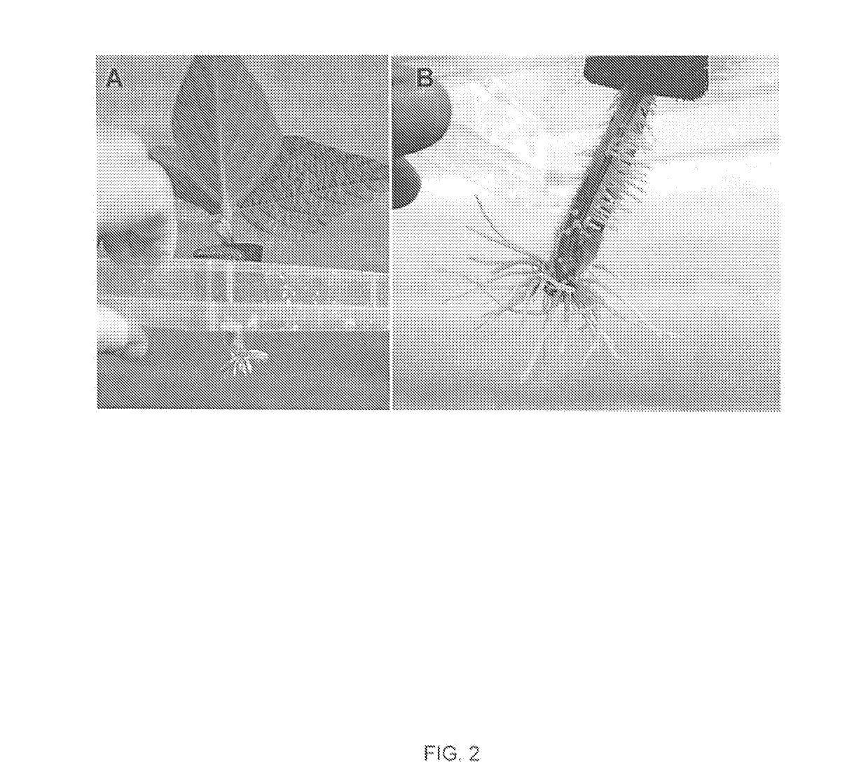

White spots started to appear on day four and these spots developed into shoot roots by day 7. Numerous roots, some of them long, were developed by day 9 (FIG. 2).

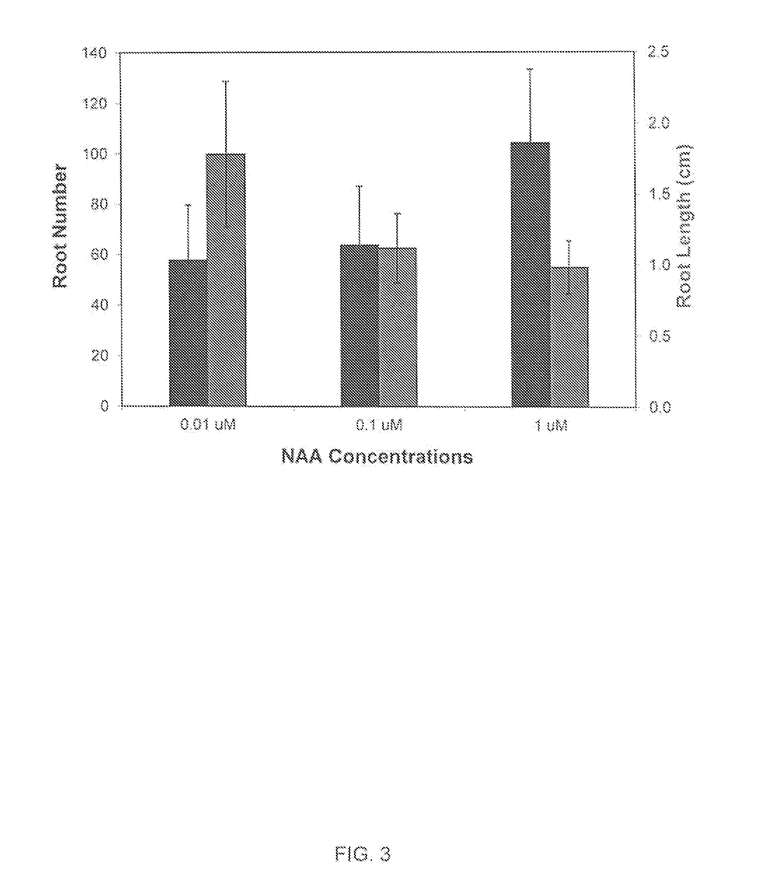

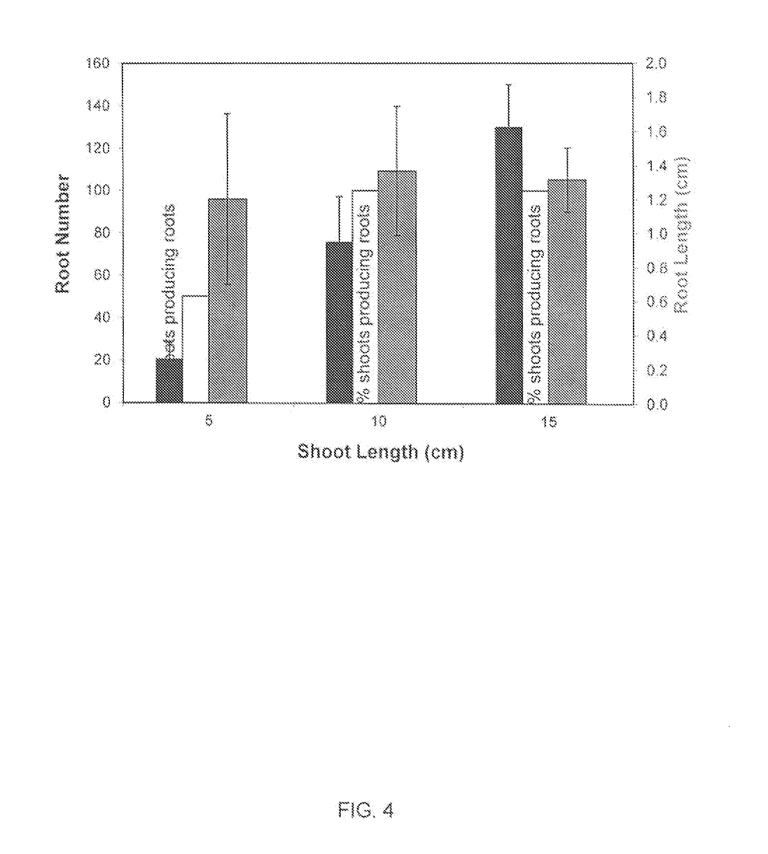

Higher NAA concentrations stimulated more roots from a shoot, but kept the roots shorter (FIG. 3). All shoots 10 cm long or longer produced roots, while 50% of shoots 5 cm long produced roots. The number of roots produced on a shoot is positively correlated with the length of the shoots, but the length of the roots were not affected by shoot length. (FIG. 4).

### Example 3

T0 and T1 soybean plants expressing a transgene were cloned as described in Example 2 with NAA at a final concentration of 1.0 μM. Shoots developed normally from the cuttings, and the plantlets were transferred at day 9 to regular potting soil (Metro Mix 340) and grown to maturity in a greenhouse. The plants grew normally at vegetative and reproductive stages and routinely produce hundreds of seed per clone. Seed were harvested from the mature plants and propagated in soil. The resulting plants also grew normally at both vegetative and reproductive stages.

### Example 4

Corn plants were grown from seeds in the greenhouse with abundant fertilizer until there were many tillers from each plant. The tillers were then removed from the plants for cloning. Tissue culture boxes were used to hold the cloning media, and lids with a 2.5 cm hole in the middle were used to hold the shoots with the help of cylindrical sponge. Fertilizer was prepared by adding 10 ml of each of fertilizer stock solutions A, B and C in 4970 ml of tap water. NAA was added at a final concentration of 10-7 M. Tillers were removed from corn plants and inserted in a cylindrical sponge roll (one tiller per roll). Each roll was inserted into the lid of a tissue culture box. The boxes were maintained in the greenhouse with 16 h light at 27° C. and 8 h dark at 21° C.

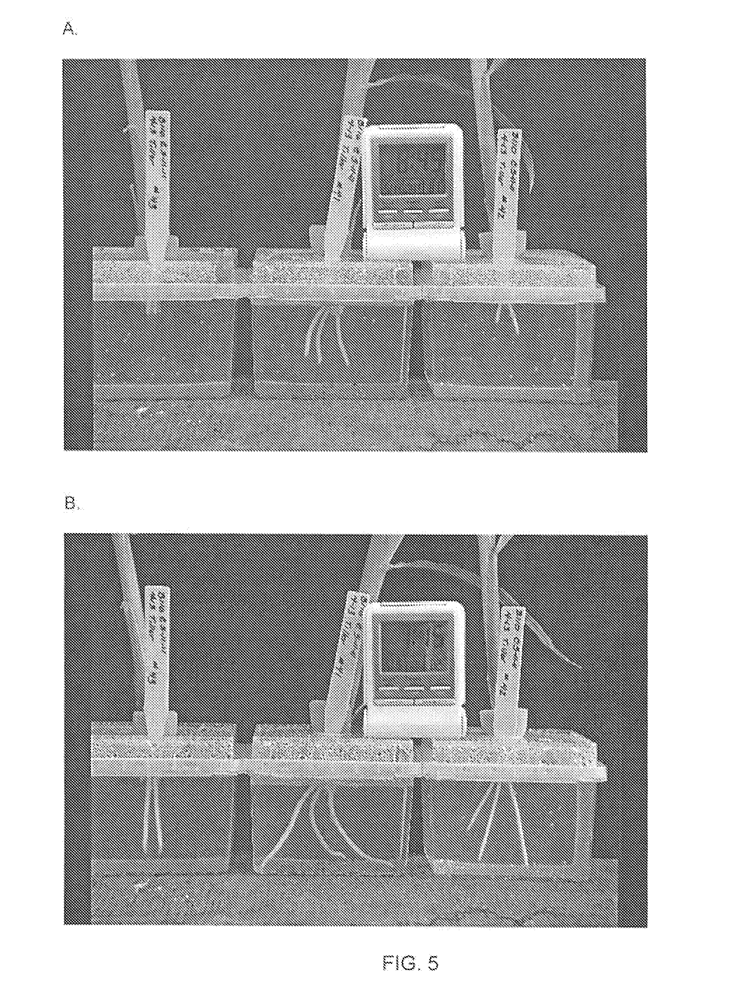

White spots began to appear on the second day of cloning and these spots developed into roots by day 3 (FIG. 5A). The roots grew rapidly and reached the bottom of the cloning box by day 5 (FIG. 5B). After transplanting to soil in pots, both the shoots and roots continued to grow. The cloned plants grew in a similar manner as the original plants from grown from seed. Fertile ear shoots and tassels developed from the cloned plants, although tassel seeds were observed on some tassels.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for the vegetative propagation of crop plants comprising removing a part of a crop plant and placing said part in a hydroponic medium sufficient to support the development of one or more roots from said part in said hydroponic medium.

2. The method of claim 1, wherein said part is a shoot.

3. The method of claim 2, wherein said shoot is at least 5 cm in length.

4. The method of claim 1, wherein said hydroponic medium contains a fertilizer.

5. The method of claim 1, wherein said hydroponic medium contains one or more plant growth hormones.

6. The method of claim 1, wherein said method comprises exposing the part of the crop plant to about 16 to about 22 hours of light per day while in the hydroponic medium.

7. The method of claim 6, wherein the light exposure is about 20 hours per day.

8. The method of claim 5, wherein said plant growth hormone is an auxin.

9. The method of claim 8, wherein said auxin is 1-Naphthaleneacetic acid (NAA).

10. The method of claim 9, wherein said auxin is applied to the hydroponic medium at a final concentration of about 10-6 to about 10-8 M.

11. The method of claim 1, wherein said hydroponic medium is contained within a translucent box, wherein said box contains a lid with an opening sufficient to support the part of the plant in an upright position in said hydroponic medium.

12. The method of claim 1, wherein the crop plant is selected from the group consisting of maize, sorghum, wheat, cotton, rice, soybean, sugarbeet, sugarcane, tobacco, barley, and oilseed rape.

13. The method of claim 11, wherein the crop plant is soybean.

14. The method of claim 11, wherein the crop plant is maize.

15. The method of claim 11, wherein the crop plant is cotton.
